(19) United States
(12) Patent Application Publication
(10) Pub. No.: US 2026/0355273 A1
(43) Pub. Date: Nov. 12, 2026

(54) COMPOSITION COMPRISING CUTS OF CEREAL STRAW

(71) Applicant: MINRIS APS, Køge (DK)

(72) Inventors: Clement Vestergaard SØRENSEN, Køge (DK); Richardt SONNICHSEN, Køge (DK)

(21) Appl. No.: 19/114,098

(22) PCT Filed: Sep. 21, 2023
(86) PCT No.: PCT/EP2023/076147

§ 371 Date: Mar. 21, 2025

(30) Foreign Application Priority Data
Sep. 23, 2022 (DK) PA 2022 00862

(51) Int. Cl. A61K 36/899; A21D 2/36; A61K 9/00; A61K 9/20; A61P 1/00; A61P 3/04

(52) U.S. Cl. CPC A61K 36/899; A21D 2/36; A61K 9/0095; A61K 9/2054; A61P 1/00; A61P 3/04

(57) ABSTRACT

The present invention relates to a cereal straw product comprising a cereal straw, wherein at least 50% of the cereal straw has a size (d50) less than 5 mm and/or wherein at least 90% of the cereal straw has a size (d90) less than 8 mm; a composition comprising the cereal straw product, including a composition for use in the treatment and prophylaxis of the human or animal body and in a food product, as a food ingredient, as a dietary supplement.

### TECHNICAL FIELD OF THE INVENTION

The present invention relates to a cereal straw product in an easily consumable form, a composition composing the product, and the composition for use the treatment of different diseases and undesirable conditions. In particular, the present invention relates to a cereal straw product having various high-value applications.

### BACKGROUND OF THE INVENTION

Cereal straw is an agricultural by-product consisting of the dry stalks of cereal plants after the grain and chaff have been removed.

Cereal straw consists mainly of cellulose, hemicelluloses, lignin, and minor components as ash and protein. These major cellulose compounds are generally indigestible, see e.g. the Scientific Opinion in EFSA Journal 2010; 8 (3): 1462: Scientific Opinion on Dietary Reference Values for carbohydrates and dietary fibre. Only some animals, particularly ruminants and termites, can digest cellulose, using symbiotic microorganisms in their guts. Humans cannot digest cellulose or cellulose compounds as these are non-digestible constituent of insoluble dietary fibre, known for acting as a hydrophilic bulking agent for faeces and potentially aiding in defecation.

The cereal straw makes up about half of the yield of cereal crops such as wheat, barley, oats, rice, rye, rice soy, and it has a number of different uses.

Until now, cereal straw has been used for e.g.:
Animal feed; where straw may be fed as part of the roughage component of the diet to cattle or horses. It has a low digestible energy and nutrient content (as opposed feeds).
Silage, where straw is fermented to increase the palatability of the straw.
Basketry; where bee skeps and linen baskets are made from coiled and bound together continuous lengths of straw. The technique is known as lip work.
Bedding: for humans or livestock. For humans, cereal straw may be used to provide straw-filled mattress, also known as a palliasse. This was common during the old days, and it is still used in many parts of the world.
Biofuels; where the cereal straw may be used as a carbon-neutral energy source and this technology is increasing rapidly, especially for the production of bio butanol. Cereal straw may be made into briquettes, which may be used as a biofuel substitute to coal.
Bioplastic; where in particular rice straw may be turned into bioplastic with mechanical properties akin to polystyrene in its dry state.
Construction material; in many parts of the world, straw is used to bind clay and concrete and cereal straw may have moderate insulation characteristics.
Thatching; where cereal straw (often wheat straw) may be used for making a waterproof, lightweight roof with good insulation properties.

Thus, the applications of cereal straw are traditionally low-cost applications and the cereal straw produce low amounts of energy when consumed or burned, and most of the applications using cereal straw are slowly disappearing.

In addition, large amounts of cereal straw (in particular wheat straw which is one of the most abundant categories of agricultural waste), are usually just abandoned and burned year after year, thus also having negative environmental impact.

Cereal straw for oral consumption is traditionally hydrolysed or enzymatic treated to make the fibre material more accessible to digestion. However, this process is often very troublesome and expensive and may result in reduced or altered effects as the organic polymer compounds are dissociated, altered and/or broken down into smaller fractions. Furthermore, the processes where the cellulose polymers, are processed using wet chemical, enzymatic, or fermentation treatments prior to or following separation processes providing separate fractions of the compounds (cellulose, hemicellulose, lignin) are very expensive and lead to fractions of non-natural composition.

WO 2020/0152178 describes a method of isolating cellulose fibres of a desired length-weighted average fibre length from cereal straw, where dewaxed, cereal straw material may be cut to length of less than 1 cm before being processed in several steps of e.g. solubilisation, separation, and aqueous suspension at basic pH.

Hence, there is a need for higher value products from cereal straw, and in particular, there is a need in the industry for higher value products from cereal straw which are more efficient and/or which are more environmentally friendly.

Thus, an object of the present invention is to provide a cereal straw product of untreated straw provided in very small lengths, which are easy for human beings to consume as dietary fibres.

It is a further object of the present invention to provide a cereal straw product having specific high value and/or environmentally friendly applications that solves the above-mentioned problems in prior art.

It is an additional object of the present invention to provide a cereal straw product of nutritional value as dietary fibre, which is essential for overall health and have significant nutritional value in other ways than digestible macro-nutrients protein, fats, and digestible carbohydrates.

Finally, it is an object to provide a cereal straw product and composition comprising the cereal straw product for use in treatment and prophylaxis of a number of human diseases and conditions, in particular related to the intestinal system.

### SUMMARY OF THE INVENTION

Thus, in its first aspect, the present invention relates to a cereal straw product comprising a cereal straw, wherein at least 50% of the cereal straw has a size (d50) less than 5 mm and/or wherein 90% of the cereal straw has a size (d90) less than 8 mm.

The cereal straw product of the present invention may be an ingestible cereal straw product. For example, the ingestible cereal straw product may be a human ingestible cereal straw product. The term “human ingestible cereal straw product” may relate to a cereal straw product that is prepared for human administration and/or is a supplement that may increase the consumption of dietary fibres. The administration may be administration of the cereal straw product as such or administration of a composition comprising the cereal straw product.

Humans and non-ruminant animals cannot digest organic compounds in the form of e.g. the indigestible cellulose, hemicellulose, and lignin compounds that mainly makes up the cereal straw material, since the gastrointestinal tract in humans and non-ruminants do not contain any enzymes that can break down the polymers to shorter chain oligomers or single monomers digestible by the human or non-ruminant. On the other hand, ruminants, such as goats, cows, hippos and sheep, can digest these organic compounds. They cope with the breakdown of the polymers with the help of symbiotic bacterial cultures and yeast fungi in the gastrointestinal system.

Thus, traditionally, to make the indigestible carbohydrates accessible to digestion by humans and non-ruminant animals, these organic compounds need to be subjected to, for example, a hydrolysing pre-treatment creating absorbable sugars. The hydrolysing pre-treatment may be enzymatic or by fermentation.

The inventors of the present invention surprisingly found that the cereal straw product according to the present invention have interesting applications when the structure of the organic compounds may be maintained, or substantially maintained, for example without using hydrolysing treatment of the cereal straw.

The inventors of the present invention surprisingly found a new cereal straw product having a high dietary value. The new cereal straw product is easily and inexpensively produced, and therefore its production has a positive impact on the environment and climate.

In its second aspect, the present invention relates to a composition comprising the cereal straw product according to the present invention.

For example, the composition may be a diet product, a flour product, an ingredient, or an absorbing product comprising the cereal straw product.

For example, the composition may be in oral or rectal administration form.

In its third aspect, the present invention relates to a tablet or capsule comprising the cereal straw product and optionally a binder ingredient.

In a 4^{th} aspect, the present invention relates to a composition comprising a cereal straw product according to the present invention, a composition according to the present invention, or a tablet or capsule according to the present invention for the treatment or prophylaxis of obesity, overweight, diarrhea, bloating, flatulence (stomach or bowel gas), inflammatory bowel disease (IBD), Crohn's disease, pain (stomach pain), high cholesterol levels, heart disease, type 2 diabetes, or colon cancer.

In a 5^{th} aspect, the present invention relates to a process for providing a cereal straw product comprising a cereal straw having a reduced size, wherein the process comprises the steps of:
(a) providing a cereal straw;
(b) subjecting the cereal straw provided in step (i) to a treatment resulting in a cereal straw product comprising cereal straw having a size (d50) less than 5 mm and/or having a size (d90) less than 8 mm.

comprising a cereal straw product for the treatment or reduction of obesity or overweight.

In a 6^{th} aspect, the present invention relates to the use of a cereal straw product of the invention in a food product, as a food ingredient, or as a dietary supplement.

In a 7^{th} aspect, the present invention relates to the cereal straw product, the composition, the tablet or the capsule for use in treatment or prophylaxis of the human or animal body.

Finally, in an 8^{th} aspect, the invention relates to a process for preparing a tablet comprising a cereal straw product according to the present invention, the process comprises the steps of:
(i) Providing a cereal straw product according to the present invention,
(ii) Adding a binder ingredient to the cereal straw provided in step (i) providing a blend (comprising the binder ingredient and the cereal straw),
(iii) Optionally, adjusting the moisture content of the blend from step (ii) to a level in the range of 30-90% (w/w) water providing a wet blend, such as in the range of 40-85% (w/w) water, e.g. in the range of 50-80% (w/w), such as in the range of 55-75% (w/w) water, e.g. in the range of 70-70% (w/w);
(iv) Optionally, mixing the wet blend to provide a homogenous blend;
(v) Pressing the homogenous blend providing the tablets comprising a cereal straw product according to the present invention.

### DETAILED DESCRIPTION OF THE INVENTION

The present invention in its first aspect relates to a cereal straw product comprising a cereal straw, wherein at least 50% of the cereal straw has a size (d50) less than 5 mm and/or wherein at least 90% the cereal straw has a size (d90) less than 8 mm.

In an embodiment of the present invention in any of its aspects, the size (d50) is in the range of 5-0.005 mm, such as in the range of 4-0.01 mm, e.g. in the range of 3-0.05 mm, such as in the range of 2-0.1 mm, e.g. in the range of 1-0.5 mm.

In yet an embodiment of the present invention in any of its aspects, the size (d90) is in the range of 8-0.005 mm, such as in the range of 5-0.01 mm, e.g. in the range of 3-0.05 mm, such as in the range of 2-0.1 mm, e.g. in the range of 1.5-0.5 mm.

In an embodiment of the invention in any of its aspects, the cereal straw is a smaller sized cereal straw, for example produced by mechanical treatment. Preferably, the smaller sized cereal straw may have a size (d50) less than 5 mm, such as less than 4 mm, e.g. less than 3 mm, such as less than 2 mm, e.g. less than 1 mm, such as less than 0.5 mm, e.g. less than 0.1 mm, such as less than 0.05 mm, e.g. less than 0.01 mm, such as less than 0.005 mm.

In another embodiment of the invention in any of its aspects, the cereal straw is a smaller sized cereal straw, for example produced by mechanical treatment. Preferably, the smaller sized cereal straw may have a size (d90) less than 8 mm, such as less than 4 mm, e.g. less than 2 mm, such as less than 1 mm, e.g. less than 0.5 mm, such as less than 0.1 mm.

In an embodiment of the invention in any of its aspects, the cereal straw product comprises less than 15% (w/w) short chain carbohydrate. Short chain carbohydrates may comprise monosaccharides and oligosaccharides of 9 or less monomeric units, 8 or less monomeric units, 7 or less monomeric units, 6 or less monomeric units, 5 or less monomeric units, 4 or less monomeric units, 3 or less monomeric units, or 2 monomeric units.

In an embodiment of the invention in any of its aspects, the cereal straw product is substantially free from cereal grains or cereal bran.

In an embodiment of the invention in any of its aspects, the cereal straw product comprises at least 95% (w/w) cellulose compounds; e.g., at least 96% (w/w) cellulose compounds, at least 97% (w/w) cellulose compounds, at least 98% (w/w) cellulose compounds, such as at least 99% (w/w) cellulose compounds.

In an embodiment of the invention in any of its aspects, the cereal straw product consists essentially of organic compounds in the form cellulose compounds, hemicellulose compounds, and lignin.

The chemical composition of cereal straw may vary depending on factors such as the specific species, the variety of cereal straw, the growth conditions, the stage of growth, environmental conditions, and other factors. However, in general, cereal straw primarily comprise cellulose, hemicellulose, proteins, minerals, lipids, ash and water.

Cereal straw, like wheat straw, barley straw, rye straw, oats straw, or triticale straw, is the most abundant indigestible biomass among agricultural residues in the world.

Cereal straw may be used mainly as feed for livestock, agricultural supplements, or is burned, thus creating environmental issues.

Cereal straw applied according to the present invention may be comprised of indigestible carbohydrates. The indigestible carbohydrates are mainly non-starch polysaccharides, like cellulose, hemicellulose, and lignin.

The indigestible carbohydrates may form part of the plant cell wall, and the cereal straw cell wall, and provides structural support and maintaining of the integrity of the cereal straw.

In an embodiment of the present invention, the indigestible carbohydrates may comprise cellulose compounds, hemicellulose compounds, and lignin.

In an embodiment of the present invention, the cereal straw product consists essentially of cellulose compounds, hemicellulose compounds, and lignin.

Lignin may provide rigidity and strength to the straw material and may act as a binding material that reinforces the structural integrity of the straw. It may also help to prevent water loss and provides protection against pathogens and pests due to its dense and resistant nature.

Cellulose compounds may form the primary structural framework of the straw material.

Cellulose compounds may provide both strength and flexibility, giving the straw material, its shape and mechanical support. Cellulose compounds may also play a role in regulating water movement within the cell wall.

Hemicellulose compounds may be a carbohydrate that surrounds cellulose fibres within the straw material. It may act as a filler material and may contribute to the overall structural integrity of the cell wall.

Lignin, cellulose compounds, and hemicellulose compounds are the three main components of cereal straw, and their arrangement and interactions may form the basis of lignocellulosic compounds.

Lignin, cellulose, and hemicellulose are indigestible carbohydrates with a degree of polymerisation.

In nature, these components (cellulose compounds, hemicellulose compounds, and lignin) may work together to support plants against environmental stress, maintain their form, and facilitate vital physiological processes.

Traditionally, the structure of the indigestible compounds is changed, and made more accessible to digestion by humans and non-ruminant animals, by subjecting the organic polymer compounds to a hydrolysing pre-treatment resulting in a hydrolysate rich in digestible sugar residues.

Humans and non-ruminant animals cannot digest the indigestible cellulose, hemicellulose, and lignin compounds since the gastrointestinal tract in humans and non-ruminants contains no enzymes that can break down the carbohydrates to shorter chain oligomers or single monomers digestible by the human or non-ruminant.

On the other hand, ruminants, such as goats, cows, hippos and sheep, can digest these organic compounds. They cope with the breakdown of the polymers with the help of symbiotic bacterial cultures and yeast fungi in the gastrointestinal system.

Thus, traditionally, to make these indigestible carbohydrates accessible to digestion by humans and non-ruminant animals, the organic compounds need to be subjected to a hydrolysing pre-treatment creating absorbable sugars. The hydrolysing pre-treatment may be enzymatic or by fermentation.

In an embodiment of the invention in all its aspects, the cereal straw may have been subjected to mechanical treatment to produce the smaller sized straw. The mechanical treatment may be shredding, cracking, cutting or milling into smaller sizes. Preferably, the mechanical treatment may be cutting. The cereal straw may be washed before the cereal straw has been subjected mechanical treatment to remove impurities. Impurities may be dust, soil, grains and/or leaves.

In an embodiment of the invention in all its aspects, the cereal straw may be subjected to mechanical treatment while the cereal straw is wet, optionally by heat water. When the wet cereal straw may be subjected to mechanical treatment the moisture content of the wet cereal straw may be in the range of 25-75% (w/w) relative to the weight of the wet cereal straw, such as in the range of 35-55% (w/w), e.g. in the range of 40-50% (w/w).

Preferably, the washing of the cereal straw may be done by adding a washing solution to the cereal straw. Preferably, the washing solution may be an aqueous washing solution. Preferably, the aqueous washing solution may be water, preferably tab water.

In an embodiment of the invention in all its aspects, the cereal straw may be subjected to drying before being subjected to mechanical treatment.

Preferably, the cereal straw product may be provided without separating one or more of the naturally occurring organic compounds present in the cereal straw. The cereal straw may comprise cellulose compounds, hemicellulose compounds, and/or lignin.

In an embodiment of the invention in all its aspects, the cereal straw may be subjected to mechanical treatment while the cereal straw is dry (either before being washed or after being subjected to drying). When the dry cereal straw may be subjected to mechanical treatment the moisture content of the dry cereal straw may be in the range of 2-25% (w/w) relative to the weight of the dry cereal straw, such as in the range of 4-20% (w/w), or in the range of 5-15% (w/w), in the range of 8-12% (w/w), e.g. in the range of 10-11% (w/w).

Preferably, the cereal straw may be subjected to an antimicrobial, a heat treatment process, a pasteurisation process or a sterilisation process to reduce or eliminate the microbial activity in the cereal straw.

In an embodiment of the invention in all its aspects, the antimicrobial process may be performed before the subjecting the cereal straw to mechanical treatment.

In an embodiment of the invention in all its aspects, the antimicrobial process may be performed after mechanical treatment and/or after size separation.

The cereal straw present in the cereal straw product according to the present invention may not be subjected to a hydrolysing pre-treatment, including a chemical treatment, an enzymatic treatment, heat treatment, pressure treatment, a fermentation treatment, or a combination hereof.

To improve the effect of the cereal straw product, the cereal straw may be selected from a cereal straw product having less than 20% (w/w) short chain carbohydrate relative to the dry weight of the cereal straw, such as less than 15% (w/w) short chain carbohydrate, e.g. less than 10% (w/w) short chain carbohydrate, such as less than 5% (w/w) short chain carbohydrate, e.g. less than 1% (w/w) short chain carbohydrate, such as 0% (w/w) short chain carbohydrate.

In the context of the present invention the short chain carbohydrates may be oligosaccharides and may comprise 1 to 45 monosaccharide units, such as 2-40 monosaccharides, e.g. 3-35 monosaccharides, such as 4-30 monosaccharides, e.g. 5-20 monosaccharides, such as 7-15 monosaccharides, e.g. 8-10 monosaccharides.

Cellulose compounds or hemicellulose compounds, according to the present invention may be considered to comprise from more than 5, more than 50, more preferably more than 75 monosaccharide units, even more preferably more than 100 monosaccharide units, even more preferably more than 200 monosaccharide units, even more preferably more than 400 monosaccharide units, even more preferably more than 600 monosaccharide units, even more preferably more than 800 monosaccharide units, even more preferably more than 1000 monosaccharide units.

In an embodiment of the invention in all its aspects, the cereal straw may be selected from the subfamily of Pooideae. The Pooideae is a subfamily of the grass family Poaceae. The subfamily Pooideae may include wheat, barley, oat, rye, triticale and various lawn and pasture grasses.

Preferably, the cereal straw selected from the subfamily of Pooideae may include wheat straw, oat straw, barley straw, rye straw, triticale straw, or a combination hereof. Preferably, the cereal straw may be selected from wheat straw, oat straw, barley straw, rye straw a combination hereof. Even more preferably, the cereal straw may be comprising wheat straw. Even more preferably, the cereal straw may consist essentially of wheat straw.

In an embodiment of the invention in all its aspects, the cereal straw product may be substantially free from cereal grains or cereal bran.

In the context of the present invention the term “substantially free from cereal grains or cereal bran” or “substantially free from cereal grains” or “substantially free from cereal bran” relates to a cereal straw product comprising less than 10% (w/w) cereal grains and/or cereal bran relative to the dry weight of the cereal straw product, such as less than 8% (w/w) cereal grains and/or cereal bran, e.g. less than 6% (w/w) cereal grains and/or cereal bran, such as less than 4% (w/w) cereal grains and/or cereal bran, e.g. less than 2% (w/w) cereal grains and/or cereal bran, such as less than 1% (w/w) cereal grains and/or cereal bran, e.g. less than 0.5% (w/w) cereal grains and/or cereal bran, such as less than 0.1% (w/w) cereal grains and/or cereal bran, e.g. no cereal grains and/or cereal bran.

In an embodiment of the invention in all its aspects, the cereal straw product consists essentially of cereal straw.

The cereal straw according to the invention in all its aspects may relate to an agricultural by-product consisting of the stalks of cereal plants after the grain and chaff have been removed.

The cereal straw product may comprise at least 50% (w/w) cereal straw relative to the dry weight of the cereal straw product, such as at least 60% (w/w), e.g. at least 70% (w/w), such as at least 75% (w/w), e.g. at least 80% (w/w), such as at least 85% (w/w), e.g. at least 90% (w/w), such as at least 95% (w/w), e.g. at least 98% (w/w), such as 100% (w/w).

In an embodiment of the present invention, the cereal straw product comprises no fruit fibres.

The cereal straw may preferably comprise a high content of the organic compounds.

The cereal straw may comprise at least 25% (w/w) organic compounds relative to the dry weight of the cereal straw; such as at least 50% (w/w) organic compounds relative to the dry weight of the cereal straw; e.g. at least 75% (w/w) organic compounds relative to the dry weight of the cereal straw; such as at least 85% (w/w) organic compounds relative to the dry weight of the cereal straw; e.g. at least 90% (w/w) organic compounds relative to the dry weight of the cereal straw; such as at least 95% (w/w) organic compounds relative to the dry weight of the cereal straw; e.g. at least 98% (w/w) organic compounds relative to the dry weight of the cereal straw; such as at least 99% (w/w) organic compounds relative to the dry weight of the cereal straw, e.g. 100% (w/w) organic compounds relative to the dry weight of the cereal straw.

In an embodiment of the invention in all its aspects, the cereal straw product comprises a high content of organic compounds, in particular non-hydrolysed organic compounds.

In a further embodiment of invention in all its aspects, the cereal straw product comprises at least 25% (w/w) organic compounds relative to the dry weight of the cereal straw; such as at least 50% (w/w) organic compounds; e.g. at least 75% (w/w) organic compounds; such as at least 85% (w/w) organic compounds; e.g. at least 90% (w/w) organic compounds; such as at least 95% (w/w) organic compounds; e.g. at least 98% (w/w) organic compounds; such as at least 99% (w/w) organic compounds, e.g. 100% (w/w) organic compounds.

The organic compounds include cellulose compounds, hemicellulose compounds, and/or lignin. Preferably, the organic compounds may be a combination of cellulose compounds, hemicellulose compounds, and lignin.

In an embodiment of the invention in all its aspects, the cereal straw product comprises 25-50% (w/w) cellulose, 20-35% (w/w) hemicelluloses, and 15-25% (w/w) lignin, all relative to the dry weight of the cereal straw. Preferably, the cereal straw product comprises 25-40% (w/w) cellulose, 20-25% (w/w) hemicelluloses, and 18-22% (w/w) lignin. Even more preferably, the cereal straw product comprises 30-35% (w/w) cellulose, 23-24% (w/w) hemicelluloses, and 19-21% (w/w) lignin.

The cereal straw product according to the invention in all its aspects may comprise less than 5% (w/w) starch determined on a dry matter basis, such as less than 4% (w/w) starch, e.g. less than 3% (w/w) starch, such as less than 2% (w/w) starch, e.g. less than 1% (w/w) starch, such as less than 0.5% (w/w) starch, e.g. less than 0.1% (w/w) starch, such as 0% (w/w) starch.

In an embodiment of the invention in all its aspects, the cereal straw product may be provided with added starch. The “added starch” may relate to a starch component or a starch source not naturally present in the cereal straw or not originating from the cereal straw used in the cereal straw product.

Additionally, the cereal straw product according to the invention in all its aspects may comprise less than 10% (w/w) crude protein determined on a dry matter basis, such as less than 8% (w/w) crude protein, e.g. less than 6% (w/w) crude protein, such as less than 5% (w/w) crude protein, e.g. less than 4% (w/w) crude protein, such as less than 3% (w/w) crude protein, e.g. less than 2% (w/w) crude protein, such as less than 1% (w/w) crude protein, e.g. less than 0.5% (w/w) crude protein, such as 0% (w/w) crude protein.

In an embodiment of the invention in all its aspects, the cereal straw product may be provided with an added crude protein. The “added crude protein” may relate to a protein source not naturally present in the cereal straw or not originating from the cereal straw used in the cereal straw product.

The cereal straw product according to the invention in all its aspects may also comprise less than 8% (w/w) crude fat determined on a dry matter basis, such as less than 6% (w/w) crude fat, e.g. less than 4% (w/w) crude fat, such as less than 2% (w/w) crude fat, e.g. less than 1.5% (w/w) crude fat, such as less than 1% (w/w) crude fat, e.g. less than 0.5% (w/w) crude fat, such as less than 0.25% (w/w) crude fat, e.g. 0% (w/w) crude fat.

In an embodiment of the invention in all its aspects, the cereal straw product may be provided with an added crude fat. The “added crude fat” may relate to a fat source not naturally present in the cereal straw or not originating from the cereal straw used in the cereal straw product.

The cereal straw product may comprise a cereal straw, wherein at least 50% of the cereal straw has a size (d50) less than 5 mm and/or wherein the at least 90% of the cereal straw has a size (d90) less than 8 mm and wherein the cereal straw product comprises less than 20% (w/w) short chain carbohydrate.

Preferably, the short chain carbohydrate may be short chain carbohydrate originating from the cereal straw used for providing the cereal straw product.

The short chain carbohydrate may be obtained from the cereal straw product from hydrolyzing, enzymatic treating or a combination hereof of the cereal straw product.

In an embodiment of the invention in all its aspects the short chain carbohydrate may comprise a single carbohydrate moiety, a linear chain of carbohydrate moieties (a part of the cellulose compound and/or the hemicellulose compound), branched chain of carbohydrate moieties (a part of the cellulose compound and/or the hemicellulose compound), or a combination hereof. Preferably, the short chain carbohydrate may comprise a single carbohydrate moiety.

Preferably, the cereal straw product comprises less than 20% (w/w) short chain carbohydrate relative to the weight of the cereal straw product, such as less than 15% (w/w) short chain carbohydrate, e.g. less than 10% (w/w) short chain carbohydrate, such as less than 5% (w/w) short chain carbohydrate, e.g. less than 1% (w/w) short chain carbohydrate, such as 0% (w/w) short chain carbohydrate.

The single carbohydrate moiety may be selected from glucose, galactose, fructose, xylose or a combination hereof. Preferably, the single carbohydrate moiety may be glucose.

In an embodiment of the invention in all its aspects, the cereal straw according to the present invention may be selected from a low content of naturally occurring carbohydrate moieties, in particular a low content of naturally occurring glucose. Preferably, the low content of naturally occurring carbohydrate moieties, in particular a low content of naturally occurring glucose, may relate to a cereal straw having a having less than 20% (w/w) naturally occurring carbohydrate moieties, in particular a low content of naturally occurring glucose, relative to the dry weight of the cereal straw, such as less than 15% (w/w), e.g. less than 10% (w/w), such as less than 5% (w/w), e.g. less than 1% (w/w), such as 0% (w/w).

In an embodiment of the invention in all its aspects, the cereal straw product may be provided with an added short chain carbohydrate. The “added short chain carbohydrate” may relate to a carbohydrate source not naturally present in the cereal straw or not originating from the cereal straw used in the cereal straw product.

The added short chain carbohydrate may comprise a single added carbohydrate moiety, a added linear chain of added carbohydrate moieties, an added branched chain of added carbohydrate moieties, or a combination hereof.

The term “(w/w)” may be intended to mean on a weight by weight basis of the specified component.

The cereal straw product may comprise more than 40 times as much carbon (C) relative to nitrogen (N) on a weight by weight basis, such as more than 50 times as much, e.g. more than 60 times as much, such as more than 70 times as much, e.g. more than 80 times as much, such as more than 90 times as much, e.g. more than 100 times as much, such as more than 125 times as much, e.g. more than 150 times as much, such as more than 200 times as much.

Preferably, the cereal straw product may comprise in the range of 40-95 times as much carbon (C) relative to nitrogen (N) on a weight by weight basis, such as in the range of 50-90 times as much, e.g. in the range of 60-80 times as much, such as in the range of 70-75 times as much.

In its second aspect, the present invention relates to a composition comprising a cereal straw product according to the present invention.

Preferably, the composition according to the present invention relates to an ingestible composition. Preferably the ingestible composition comprising an ingestible cereal straw product.

In an embodiment of the present invention, the ingestible composition and/or the ingestible cereal straw product may be a product provided for human or animal administration. Preferably, the animal administration may be non-ruminant administration. Preferably, the animal administration may be pet administration. Preferably, the animal administration may be non-ruminant pet administration.

Human administration may include oral consumption and/or rectal administration. Preferably, human administration may include oral consumption.

In an embodiment of the present invention the composition may comprise in the range of 0.5-100% (w/w) of the cereal straw product relative to the dry weight of the composition, such as in the range of (0.75-95% (w/w), e.g. in the range of 1-85% (w/w), such as in the range of (1.25-75% (w/w), e.g. in the range of 1.5-50% (w/w), such as in the range of 1.75-25% (w/w), e.g. in the range of 2-15% (w/w), such as in the range of 2.5-10% (w/w), e.g. in the range of 3-8% (w/w), such as in the range of 3.5-6% (w/w), e.g. in the range of 4-5% (w/w).

Preferably, the composition according to the present invention may have a moisture content in the range of 1-15% (w/w) relative to the weight of the cereal straw, such as in the range of 1-12% (w/w), e.g. in the range of 2-10% (w/w), such as in the range of 4-8% (w/w), e.g. in the range of 5-7% (w/w).

In an embodiment of the present invention, the composition may be a product for human consumption or human administration. Preferably, the composition according to the present invention may be a composition for oral consumption or for rectal consumption. Even more preferably, the composition according to the present invention may be a human oral composition.

The composition according to the present invention may be subjected to one or more further treatments after being mixed with the cereal straw product.

In an embodiment of the invention in all its aspects, the cereal straw product may be present in the composition as a free-flowing ingredient. The free-flowing ingredients may be ingredients that do not stick to each other and there may not be any (or substantially no) aggregate formation between the cereal straw products. Preferably, the free-flowing ingredient may be an ingredient, which do not stick to other ingredients (ingredients different from the cereal straw product) in the composition, preferably, other main ingredients, and thereby form any aggregates. The other ingredients may include minerals, small ions, and the like.

In an embodiment of the present invention, the composition may be a diet product, a flour product, an ingredient, and/or an absorbing product.

A preferred embodiment of the present invention relates to a diet product comprising a cereal straw product.

In an embodiment of the present invention the diet product comprising the cereal straw of the cereal straw product may have a size (d50)—of the cereal straw—being less than 5 mm, such as less than 4 mm, e.g. less than 3 mm, such as less than 2 mm, e.g. less than 1 mm, such as less than 0.5 mm, e.g. less than 0.1 mm, such as less than 0.05 mm, e.g. less than 0.01 mm, such as less than 0.005 mm.

Furthermore, the cereal straw—of the cereal straw product—may have a size (d90) less than 8 mm, such as less than 4 mm, e.g. less than 2 mm, such as less than 1 mm, e.g. less than 0.5 mm, such as less than 0.1 mm.

In an embodiment of the invention in all its aspects, the cereal straw—of the cereal straw product—may have a size (d50) less than 5 mm, such as less than 4 mm, e.g. less than 3 mm, such as less than 2 mm, e.g. less than 1 mm, such as less than 0.5 mm, e.g. less than 0.1 mm, such as less than 0.05 mm, e.g. less than 0.01 mm, such as less than 0.005 mm, and a size (d90) less than 8 mm, such as less than 4 mm, e.g. less than 2 mm, such as less than 1 mm, e.g. less than 0.5 mm, such as less than 0.1 mm.

In an embodiment of the present invention the diet product comprises in the range of 10-100% (w/w) cereal straw product in the diet product relative to the dry weight of the diet product, such as in the range of 20-98% (w/w) cereal straw product, such as in the range of 30-96% (w/w) cereal straw product, e.g. in the range of 50-95% (w/w) cereal straw product, such as in the range of 65-92% (w/w) cereal straw product, e.g. in the range of 75-90% (w/w) cereal straw product, such as in the range of 80-85% (w/w) cereal straw product.

Preferably, the diet product may be a human diet product.

In an embodiment of the present invention, the diet product may be provided for oral consumption.

The diet product according to the present invention may be suitable for reducing the body weight of a human.

In its 6^{th} aspect, the present invention relates to the use of a cereal straw product of the invention in a food product, as a food ingredient, or as a dietary supplement.

In its 7^{th} aspect, the present invention relates to the cereal straw product of the invention or the composition of the invention for use in the treatment or prophylaxis of the human or animal body (such as for the treatment or prevention of an intestinal induced diseases).

A preferred embodiment of the present invention relates to a diet product (and/or a composition) comprising a cereal straw product for the treatment or reduction of obesity or overweight.

Obesity may be a condition in which body fat (sometimes excess body fat) has accumulated to an extent that may have a negative effect on health. Obesity in accordance with the present invention may also relate to conditions in which body fat has accumulated to an extent that is undesirable, but not necessarily relates to health issues.

Preferably, obesity may be a condition in which excess body fat has accumulated to an extent that may have a negative effect on health.

The diet product (and/or a composition) according to the present invention comprising a cereal straw product for the treatment or reduction of a human suffering from obesity.

In an embodiment of the present invention, the person suffering from obesity may be a person having a body mass index (BMI) above 30, such as a BMI above 32, e.g. a BMI above 35, such as a BMI above 37, e.g. a BMI above 40.

A preferred embodiment of the present invention relates to a composition comprising a cereal straw product for the treatment of intestinal induced diseases and/or normalisation of the intestinal microbiota.

The composition according to the present invention may be suitable for the treatment of malfunctioned intestine and/or treatment of intestinal induced diseases.

In an embodiment of the present invention the malfunctioned intestine and/or treatment of intestinal induced diseases may relate to obesity, overweight, diarrhea, inflammatory bowel disease (IBD), Crohn's disease, pain (stomach pain), bloating, flatulence, reduced nutritional uptake, reduce the cholesterol levels, improve blood sugar control, reduce the risk of heart disease, reduce the risk of type 2 diabetes, or to reduce the risk of colon cancer.

In an embodiment of the present invention, the malfunctioned intestine and/or treatment of intestinal induced diseases may demonstrate a stool type 1-2 or 5-7 according to the Bristol Stool Chart, whereas a normal functional intestine may demonstrate a stool type 3-4.

In an embodiment of the present invention, the malfunction may demonstrate constipation and defecation every third day or less.

In an embodiment of the present invention, the malfunction may demonstrate diarrhea as loose, watery stools three or more times a day.

The composition according to the present invention may be supplemented with one or more probiotic microorganisms or it may not be supplemented with a probiotic microorganism.

When the composition according to the present invention may be supplemented with one or more probiotic microorganisms, preferably, the one or more probiotic microorganisms may be selected from one or more lactic acid bacterium, Bifidobacterium, or a combination hereof.

In an embodiment of the present invention, the composition according to the present invention is not supplemented with microorganisms. Preferably, the composition according to the present invention does not comprise microorganisms.

Preferably, the amount of cereal straw product administered on a daily basis may be in the range of 1-50 g cereal straw product per day, such as in the range of 3-40 g per day, e.g. 5-30 g per day, such as in the range of 7-20 g per day, e.g. 9-15 g per day, such as in the range of 11-13 g per day.

In an embodiment of the present invention, the composition according to the present invention or the diet product according to the present invention may be formulated as a powder, as a capsule or as a tablet. Preferably, the composition or the diet product may be formulated as a capsule or a tablet.

In an embodiment of the present invention the composition according to the present invention or the diet product may be provided as a capsule or a tablet having a unit dose (per capsule/tablet) in the range of 0.25-10 g cereal straw product per capsule/tablet, such as in the range of 0.5-8 g cereal straw product per capsule/tablet, e.g. in the range of 1-7 g cereal straw product per capsule/tablet, such as in the range of 2-6 g cereal straw product per capsule/tablet, e.g. in the range of 3-5 g cereal straw product per capsule/tablet.

The inventors of the present invention surprisingly found that a daily administration of the composition according to the present invention or the diet product according to the present invention, a slight overweight human, having a body mass index value (BMI-value) in the range of 28-35 may lose about one kg per week. Thus, the administration of the diet product according to the present invention showed promising in the treatment of obesity (or overweight) without traditional side effects, like nausea, vomiting, and/or constipation.

The inventors found that the defecation normalized, being consistency and regularity. Additionally that flatulence and feeling of bloating reduced.

The inventors of the present invention found that satiety increase and time to feeling hungry increased. Additionally, the size of meal needed to the feeling of full was reduced.

Without being bound by theory, the inventor of the present invention trust that the reason traditional side effects may be avoided by administering the composition or the diet product according to the present invention may be because the diet product does not, or substantially not, swell when taken up liquid in the stomach of the human.

Additionally the inventors of the present invention found that the intake of dietary fibre is increased without additional calorie intake and/or without simultaneously intake of additional volume food to reach the same intake of dietary fibre.

In an embodiment of the present invention the composition according to the present invention or the diet product of the present invention may comprise a cereal straw product be as defined herein.

In an embodiment of the present invention, the composition according to the present invention or the diet product may be supplemented with a flavouring agent.

Flavouring agents well-known in the food and confection art may be added to the composition or the diet product of the instant invention. These flavouring agents may be chosen from synthetic flavour oils and/or those derived from natural fruits, plants, leaves, flowers, and combinations thereof. Representative flavours are artificial, natural or synthetic fruit flavours, such as citrus oil, including lemon, orange, grape, lime and grapefruit and fruit essences including apple, apricot, strawberry, cherry, pineapple and so forth. Also useful are flavour oils such as spearmint oil, cinnamon oil, oil of wintergreen (methylsalicylate) and peppermint oils.

The amount of flavouring agent employed may normally be a matter of preference, subject to such factors as flavour type, base type and strength desired. In general, amounts in the range of 0.05-5% (w/w) of the total diet composition relative to the dry weight of the diet composition may be useable, such as in the range of 0.3-2.5% (w/w), e.g. in the range of 0.7-2.0% (w/w), such as in the range of 1.0-1.5% (w/w).

In its third aspect, the present invention relates to a tablet or capsule comprising the cereal straw product according to the present invention.

In addition to the cereal straw product according to the present invention, the tablet may be provided with a binder ingredient. The binder ingredient may be provided to hold to the components of the tablet together and improving physical stability of the tablet.

In an embodiment of the present invention the tablet may comprise less than 20% (w/w) of the binder ingredient relative to the dry weight of the tablet, such as less than 15% (w/w) binder ingredient, e.g. less than 10% (w/w) binder ingredient, such as less than 5% (w/w) binder ingredient, e.g. less than 3% (w/w) binder ingredient, such as in the range of 0.1-20% (w/w) of the binder ingredient, e.g. in the range of 0.5-15% (w/w) binder ingredient, such as in the range of 1-10% (w/w) of the binder ingredient, e.g. in the range of 3-5% (w/w) binder ingredient.

A preferred binder ingredient may be selected from carboxymethyl cellulose (CMC), microcrystalline cellulose (MCC) or silicified microcrystalline cellulose (SMCC). CMC, MCC and/or SMCC may be a partially depolymerized cellulose product synthesized from a-cellulose precursor. The SMCC may comprise a mixture of MCC and colloidal silicon dioxide.

Preferably, the silicified microcrystalline cellulose may comprise MCC up to 98% (w/w) and the remaining 2% (w/w)—or higher—colloidal silicon dioxide. Preferably, the SMCC comprises 85-99% (w/w) MCC and 1-15% (w/w) colloidal silicon dioxide, more preferably, in the range of 95-98% (w/w) MCC and 2-5% (w/w) colloidal silicon dioxide, even more preferably about 98% (w/w) MCC and 2% (w/w) colloidal silicon dioxide.

In some applications, SMCC may be the preferred binder since it may improve binding capability in the tablet formulations. This may cause the amount of binder ingredient used may be reduced when using SMCC compared to other binders, e.g. like MCC. In addition, SMCC may also show improved resistance to the degrading effects of magnesium stearate compared with other binder ingredients.

Preferably, the CMC or the MCC may have a degree of polymerization less than 400, such as less than 300, e.g. less than 200, such as less than 100, e.g. in the range of 25-400, such as in the range of 50-375, e.g. in the range of 100-350, such as in the range of 150-300, e.g. in the range of 200-250.

In an embodiment of this aspect of the present invention less than 50% of the binder ingredient, e.g. the CMC or MCC particles, such as less than 40%, e.g. less than 30%, such as less than 20%, e.g. less than 10%, such as less than 5%; has a particle size lower than 50 μm, e.g. less than 40 μm, such as less than 30 μm, e.g. less than 20 μm, such as less than 10 μm e.g. less than 5 μm, such as less than 3 μm, e.g. less than 1 μm, such as less than 0.1 μm.

Different processes such as reactive extrusion can synthesize the MCC, enzyme mediated, mechanical grinding, ultrasonication, steam explosion and acid hydrolysis. The later process can be done using mineral acids such as H₂SO₄, HCl and HBr as well as ionic liquids. The role of these reagents is to destroy the amorphous regions leaving the crystalline domains.

A preferred embodiment of the present invention in its second aspect relates to a flour product comprising a cereal straw product.

The inventors of the present invention surprisingly found that the cereal straw product according to the present invention may be suitable as a flour additive, full or partly flour replacer. In this why it is possible to exchange part of the flour with cheap cereal straw product without significantly compromising the properties of the flour product, e.g. during baking bread and introducing an increased fibre content of flour product and improved health effects.

The inventor of the present invention trusts that the fibres of the cereal straw product according to the present invention may have the effect of keeping food moving through the human gut and the rest of the digestive tract by helping to push waste through the body.

When cereal straw product gets to the large intestine, the microflora in the gut helps to ferment, or partly ferment, the fibre with the generation of e.g. sugars, short-chain fatty acids, methane, hydrogen, and carbon dioxide. Continuous supplementation of cereal straw products may be expected to lead to noticeable increase in stool output and a shortened transit time for waste.

Thus, the cereal straw product may be considered to have the ability to help support gut health in humans, especially when it comes to large intestine.

In an embodiment of the present invention the flour product comprises in the range of 0.5-10% (w/w) cereal straw product in the flour product, such as in the range of 0.75-5% (w/w) cereal straw product, e.g. in the range of 1-3% (w/w) cereal straw product, such as in the range of 1.25-2% (w/w) cereal straw product.

The cereal straw product added to the flour product may comprise cereal straw having a size (d50)—of the cereal straw—being less than 5 mm, such as less than 4 mm, e.g. less than 3 mm, such as less than 2 mm, e.g. less than 1 mm, such as less than 0.5 mm, e.g. less than 0.1 mm, such as less than 0.05 mm, e.g. less than 0.01 mm, such as less than 0.005 mm, e.g. less than 0.001 mm, such as less than 0.0005 mm, e.g. less than 0.0001 mm.

Furthermore, the cereal straw product added to the flour product having a size (d90)—of the cereal straw—being less than 8 mm, such as less than 4 mm, e.g. less than 2 mm, such as less than 1 mm, e.g. less than 0.5 mm, such as less than 0.1 mm, e.g. less than 0.05 mm, such as less than 0.01 mm, e.g. less than 0.005 mm.

In an embodiment of the present invention the cereal straw product added to the flour product having a size (d50)—of the cereal straw—being less than 5 mm, such as less than 4 mm, e.g. less than 3 mm, such as less than 2 mm, e.g. less than 1 mm, such as less than 0.5 mm, e.g. less than 0.1 mm, such as less than 0.05 mm, e.g. less than 0.01 mm, such as less than 0.005 mm, e.g. less than 0.001 mm, such as less than 0.0005 mm, e.g. less than 0.0001 mm, and a size (d90) less than 8 mm, such as less than 4 mm, e.g. less than 2 mm, such as less than 1 mm, e.g. less than 0.5 mm, such as less than 0.1 mm, e.g. less than 0.05 mm, such as less than 0.01 mm, e.g. less than 0.005 mm.

In an embodiment of the present invention, the cereal straw product when used in the flour product of the present invention may be as defined herein.

The flour product according to the present invention may comprise wheat flour, rye flour, barley flour, oat flour, soy flour, or a combination hereof. Preferably, the flour may be wheat flour.

In an embodiment of the present invention, the flour product comprises a high fibre content. Preferably, the fibre content of the flour product may be in the range of 8-15% (w/w), such as in the range of 10-14% (w/w), e.g. in the range of 12-13% (w/w).

The present invention relates to a bread comprising a cereal straw product. Preferably, the bread comprises a cereal straw product according to the present invention.

An advantage of substituting part of the flour in e.g. bread with cereal straw products according to the present invention may be that a cheaper bread may be produced, the CO₂ footprint is reduced as the cereal straw is considered a waste material that needs to be disposed, and the glycemic index of bread, in particular sourdough bread, may be reduced, resulting in consumers may obtain a health benefit from consuming bread comprising the flour product and the cereal straw product according to the present invention. All this without sacrificing the physical and/or sensorial properties of the bread. At some points the sensorial properties of the bread, such as the aroma and taste of the bread may be stronger and/or the umami taste may be improved.

In an embodiment of the present invention the glycemic index of a flour product, such as a bread, may be reduced by at least 1% relative to a similar flour product, such as a similar bread, without the cereal straw product according to the present invention, such as at least 3%, e.g. at least 5%, such as at least 10%, such as at least 15%, e.g. at least 20%, such as at least 30%, such as at least 40%, e.g. at least 50%, such as in the range of 1-50%, e.g. in the range of 3-40%, such as in the range of 5-30%, e.g. in the range of 7-20%, such as in the range of 10-15%.

In an embodiment of the present invention the shelf-life of the flour product, in particular a bread product, may be increasing with increasing content of cereal straw product in the bread dough. Preferably, the shelf-life of the flour product may be increased by at least 10%, such as by at least 25%, such as by at least 50%, e.g. by at least 75%, such as by at least 100%.

A preferred embodiment of the present invention relates to an absorbing product comprising a cereal straw product.

The cereal straw product added to the absorbing product may comprise cereal straw having a size (d50)—of the cereal straw—being less than 5 mm, such as less than 4 mm, e.g. less than 3 mm, such as less than 2 mm, e.g. less than 1 mm, such as less than 0.5 mm, e.g. less than 0.1 mm, such as less than 0.05 mm, e.g. less than 0.01 mm, such as less than 0.005 mm.

Furthermore, the cereal straw product added to the absorbing product having a size (d90)—of the cereal straw—being less than 8 mm, such as less than 4 mm, e.g. less than 2 mm, such as less than 1 mm, e.g. less than 0.5 mm, such as less than 0.1 mm.

In an embodiment of the present invention the cereal straw product added to the absorbing product having a size (d50)—of the cereal straw—being less than 5 mm, such as less than 4 mm, e.g. less than 3 mm, such as less than 2 mm, e.g. less than 1 mm, such as less than 0.5 mm, e.g. less than 0.1 mm, such as less than 0.05 mm, e.g. less than 0.01 mm, such as less than 0.005 mm, and a size (d90) less than 8 mm, such as less than 4 mm, e.g. less than 2 mm, such as less than 1 mm, e.g. less than 0.5 mm, such as less than 0.1 mm.

In an embodiment of the present invention, the cereal straw product when used in the absorbing product of the present invention may be as defined herein.

In an embodiment of the present invention the absorbing product comprises in the range of 60-100% (w/w) cereal straw product in the absorbing product relative to the dry weight of the absorbing product, such as in the range of 75-98% (w/w) cereal straw product, e.g. in the range of 85-95% (w/w) cereal straw product, such as in the range of 90-92% (w/w) cereal straw product.

Preferably, the absorbing product according to the present invention may be suitable for absorbing a hydrophobic compound, preferably, an organic compound.

The absorbing product according to the present invention may be suitable for absorbing a hydrophobic compound from any surface. Preferably, the absorbing product may be absorbing the hydrophobic compound from the skin of a human or animal body.

Preferably, the hydrophobic compound may be an organic compound.

In an embodiment of the present invention the organic compound may be a vegetable fat or oil, an animal fat or oil or a hydrocarbon compound.

An embodiment of the present invention relates to a skin cleaning product comprising (consists essentially of) the absorbing product according to the present invention.

During use, a human having a hydrophobic compound, e.g. a vegetable oil or a hydrocarbon, on the hands, may “wash” hands with a suitable amount of the skin cleaning product according to the present invention. The absorbing product of the skin cleaning product may absorb the hydrophobic compound and remove it from the hands.

In an embodiment of the present invention the skin cleaning product (and the absorbing product) may absorb at least 10% (w/w) of the hydrophobic compound relative to the weight of the skin cleaning product (and the absorbing product), such as at least 25% (w/w); e.g. at least 50% (w/w), such as at least 75% (w/w); e.g. at least 100% (w/w), such as at least 125% (w/w); e.g. at least 150% (w/w).

An advantage of the absorbing product and the absorption of organic compounds, like vegetable fat or oil, an animal fat or oil or a hydrocarbon compound, may be that the environmental impact from discharging such organic compounds to the cleaning stations and into the environment may be significantly reduced.

In an embodiment of the present invention the absorbing product according to the present invention may absorb organic compounds from land, or from a water surface. Preferably, the land may be the street or a field. Preferably, the water surface may be a sea water surface or a lake water surface.

During use for absorbing organic compounds from land or from water, the absorbing product may be placed on top of the organic compounds and allowed to absorb for a period of time. When sufficient amount of the organic compounds has been absorbed, the absorbing product (comprising the organic compounds) may be collected. The collected absorbing product may be burnt or the organic compounds may be isolated from the absorbing product.

In an embodiment of the present invention the absorbing product may absorb at least 10% (w/w) of the organic compound relative to the weight of the absorbing product, such as at least 25% (w/w); e.g. at least 50% (w/w), such as at least 75% (w/w); e.g. at least 100% (w/w), such as at least 125% (w/w); e.g. at least 150% (w/w).

In its 4^{th} aspect, the present invention relates to a process for providing a cereal straw product comprising a cereal straw having a reduced size, wherein the process comprises the steps of:
(a) providing a cereal straw;
(b) subjecting the cereal straw provided in step (a) to a mechanical treatment resulting in a cereal straw product comprising cereal straw having a size (d50) less than 5 mm and/or having a size (d90) less than 8 mm.

The process according to the present invention does not involve a hydrolysing pre-treatment of the cereal straw.

In an embodiment of the present invention the hydrolysing pre-treatment may be selected from a chemical treatment, an enzymatic treatment, heat treatment, pressure treatment, a fermentation treatment, or a combination hereof.

A preferred embodiment of the present invention relates to a process for providing a cereal straw product comprising a cereal straw having a reduced size, wherein the process consists essentially of:
(a) providing a cereal straw;
(b) subjecting the cereal straw provided in step (a) to a mechanical treatment resulting in a cereal straw product comprising cereal straw having a size (d50) less than 5 mm and/or having a size (d90) less than 8 mm.

The term “consists essentially of” may relate to a limitation of the scope of a feature or a claim to the specified features or steps, and those features or steps, not mentioned and which do not materially affect the basic and novel characteristic(s) of the claimed invention.

In an embodiment of the present invention the cereal straw product comprising a reduced size cereal straw may be subjected to a size fractionation process

The skilled person would know various ways to separate the reduced size cereal straw provided in step (b) to the desired sizes by the size fractionation process, in the present method the desired size a size (d50) less than 5 mm. A preferred size fractionation process may be sieving process providing the desired product sizes.

Preferably, the size fractionation process may result in a cereal straw product having a size (d50)—of the cereal straw—being less than 5 mm, such as less than 4 mm, e.g. less than 3 mm, such as less than 2 mm, e.g. less than 1 mm, such as less than 0.5 mm, e.g. less than 0.1 mm, such as less than 0.05 mm, e.g. less than 0.01 mm, such as less than 0.005 mm, e.g. less than 0.001 mm, such as less than 0.0005 mm, e.g. less than 0.0001 mm.

Furthermore, the cereal straw—of the cereal straw product obtained—may have a size (d90) less than 8 mm, such as less than 4 mm, e.g. less than 2 mm, such as less than 1 mm, e.g. less than 0.5 mm, such as less than 0.1 mm, e.g. less than 0.05 mm, such as less than 0.01 mm, e.g. less than 0.005 mm.

In an embodiment of the present invention the cereal straw—of the cereal straw product—may have a size (d50) less than 5 mm, such as less than 4 mm, e.g. less than 3 mm, such as less than 2 mm, e.g. less than 1 mm, such as less than 0.5 mm, e.g. less than 0.1 mm, such as less than 0.05 mm, e.g. less than 0.01 mm, such as less than 0.005 mm, e.g. less than 0.001 mm, such as less than 0.0005 mm, e.g. less than 0.0001 mm, and a size (d90) less than 8 mm, such as less than 4 mm, e.g. less than 2 mm, such as less than 1 mm, e.g. less than 0.5 mm, such as less than 0.1 mm, e.g. less than 0.05 mm, such as less than 0.01 mm, e.g. less than 0.005 mm.

The process of providing the cereal straw product may comprise a step of washing. The cereal straw may be washed before or after the cereal straw has been subjected mechanical treatment to remove impurities, like dust, soil, grains and/or leaves.

In an embodiment of the present invention, the cereal straw may be subjected to mechanical treatment while the cereal straw is wet. When the wet cereal straw may be subjected to mechanical treatment the moisture content of the wet cereal straw may be in the range of 25-75% (w/w) relative to the weight of the wet cereal straw, such as in the range of 35-55% (w/w), e.g. in the range of 40-50% (w/w).

Preferably, the step of washing the cereal straw may be done by adding a washing solution to the cereal straw. Preferably, the washing solution may be an aqueous washing solution. Preferably, the aqueous washing solution may be water, preferably tab water

In an embodiment of the present invention, the cereal straw may be subjected to step of drying before being subjected to mechanical treatment.

In an embodiment of the present invention, the cereal straw may be subjected to mechanical treatment while the cereal straw is dry (either before being washed or after being subjected to drying). When the dry cereal straw may be subjected to mechanical treatment the moisture content of the dry cereal straw may be in the range of 0-10% (w/w) relative to the weight of the dry cereal straw, such as in the range of 4-8% (w/w), e.g. in the range of 5-7% (w/w).

Preferably, the cereal straw may be subjected to an antimicrobial process, a heat treatment, a pasteurisation process or a sterilisation process to reduce or eliminate the microbial activity in the cereal straw.

In an embodiment of the present invention the antimicrobial process, the heat treatment, the pasteurisation process or the sterilisation process may be performed before the subjecting the cereal straw to mechanical treatment.

In a preferred embodiment the antimicrobial process, a heat treatment, a pasteurisation process or a sterilisation process may be performed after subjecting the cereal straw to size fractionation step.

In its 5^{th} aspect, the present invention relates to a process for preparing a tablet comprising a cereal straw product according to the present invention, the process comprises the steps of:
(i) Providing a cereal straw product according to the present invention,
(ii) Adding a binder ingredient to the cereal straw provided in step (i) providing a blend (comprising the binder ingredient and the cereal straw);
(iii) Optionally, adjusting the moisture content of the blend from step (ii) to a level in the range of 30-90% (w/w) water providing a wet blend, such as in the range of 40-85% (w/w) water, e.g. in the range of 50-80% (w/w), such as in the range of 55-75% (w/w) water, e.g. in the range of 70-70% (w/w);
(iv) Optionally, mixing the wet blend to provide a homogenous blend;
(v) Pressing the homogenous blend providing the tablets comprising a cereal straw product according to the present invention.

In an embodiment of the present invention, the wet mixture obtained in step (iv) may be subjected to drying before the tablets are pressed providing a dried blend.

In another embodiment of the present invention the step of adjusting the moisture content (step (iii)) and the step of mixing the wet mixture (step (iv)) may be omitted and the blend from step (ii) may directly (without the adjusting the water content, e.g. without addition of water), be subjected to pressing of the tablets as described in step (v), resulting in the tablet comprising a cereal straw product according to the present invention.

The dried blend may comprise a moisture content below 15% (w/w) water, such as below 10% (w/w) water, e.g. below 8% (w/w) water, such as below 6% (w/w) water, e.g. below 5% (w/w) water, such as below 4% (w/w) water, e.g. below 3% (w/w) water.

The dried blend may be milled before pressed to tablets, providing a milled blend.

In an embodiment of the present invention the milled blend may comprise a particle size in the range of 50-2000 μm, such as in the range of 75-1500 μm, e.g. in the range of 100-1000 μm, such as in the range of 150-850 μm, e.g. in the range of 200 750 μm, such as in the range of 250-600 μm.

In another embodiment of the present invention the tablet comprises a cereal straw, wherein at least 50% of the cereal straw has a size (d50) less than 5 mm and/or wherein the cereal straw has a size (d90) less than 8 mm.

In a further embodiment according to the present invention, a lubricant may be added. Preferably, the lubricant may be added to the blend, to the wet blend, to the homogenous blend, the dried blend and/or to the milled blend (if the dried blend is milled), providing a blend comprising lubricant.

The lubricants useful in the present invention are among those well-known in the art. Representative examples of such lubricants include stearic acid, metallic stearates, hydrogenated vegetable oils, partially hydrogenated vegetable oils, animal fats (e.g. triglycerides), modified maltodextrins, polyethylene glycol, light mineral oil, sodium benzoate and mixtures thereof.

Among these lubricants, stearic acid, hydrogenated vegetable oils, partially hydrogenated vegetable oils, animal fats and modified maltodextrins may be preferred.

The amount of lubricant added may vary with the specific lubricant selected and with the cereal straw being tableted. Generally, the amount of lubricant added may be about 0.01-5% (w/w) lubricant relative to the blend comprising lubricant on a dry matter basis, preferably about 0.05-3.5% (w/w), even more preferably, about 0.5-2.5% (w/w).

Tablets may be defined according to their method of manufacture. The preferred methods of manufacturing tablets may include molded tablets or compressed tablets.

Molded tablets, originally made from moist materials in a triturate, mold, and may be made on table machines, generally in small scale operations.

Compressed tablets, on the other hand, may be the most widely used dosage form since they are convenient to use, deliver the intended dose with a high degree of accuracy, and are capable of being produced by large scale processes. Among the methods for preparing compressed tablets, the wet granulation method is the most popular due to the increased probability that the granulation will meet all the physical requirements for compression into good tablets.

The tablets according to the present invention may comprise a sufficient strength to withstand handling and shipping prior to use. They may preferably be free from defects such as cracks and/or chipped edges. The tablets may preferably comprise reasonable storage stability.

In an embodiment of the present invention the tablets according to the present invention the ability to release the cereal straw of the cereal straw product in a reproducible and predictable manner.

The tablet according to the present invention may be a molded tablet or compressed tablet. Preferably, the tablet according to the present invention may be a compressed tablet.

Preferably, the amount of binder ingredient added to the cereal straw may constitute in the range of 1-30% (w/w) of the blend on a dry matter basis, such as in the range of 5-25% (w/w), e.g. in the range of 10-20% (w/w).

In an embodiment of the present invention, the tablet may be a swallowable tablet or a chewable tablet. Preferably, the tablet may be a swallowable tablet.

It should be noted that embodiments and features described in the context of one of the aspects or one embodiment of the present invention also apply to the other aspects or other embodiments of the invention.

The invention will now be described in further details in the following non-limiting examples.

### EXAMPLES

### Example 1

### Investigating the Effects of a Cereal Straw Product According to the Present Invention on Weight Loss.

Test-tablets comprising 1 g wheat straw product having a particle size d50 of about 1 mm were produced by using only mechanical treatment of the wheat straw by cutting and milling, no hydrolysis and no enzymatic treatment was used. The wheat straw product comprised less than 1% (w/w) short chain carbohydrates (relative to the total wheat straw product).

The test-tablets were produced by dry mixing the straw with CMC in the range of 10:1 to 20:1 and pressed on a Diaf Tablet Compressing machine, type T.M. spec.

The treatment was performed as a cross-over test design in a period of 2 times 2 months starting with a placebo tablet followed by a second period with test tablets. The following results were observed.

| | | 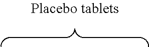 | | | 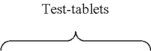 | | |
|---|---|---|---|---|---|---|---|
| | | Weight | Weight | Number | Weight | Weight | |
| | Hight | before | after | tablets | before | after | Number |
| Participant | (cm) | (kg) | (kg) | (Per day) | (kg) | (kg) | tablets |
| 1 | 173 | 81 | 80 | 12 | 80 | 75 | 12 |
| 2 | 168 | 73 | 72 | 12 | 72 | 67 | 12 |
| 3 | 168 | 70 | 71 | 12 | 71 | 68 | 12 |

The results show that the tablets comprising 1 g wheat straw product having a particle size d50 of about 1 mm resulted in a weight loss of about 4-7% within 2 months.

The administration also resulting in all participants experienced the desire for sugar and candy disappeared and the tendency of feeling hungry was reduced and digestion was improved. Two of the participants experienced a relive in constipation-like symptoms.

Furthermore, none of the participants experienced any nauseas. It is expected that the number of tablets administered may be high and the period may be long without introducing nauseas and swollen feelings because the cereal straw product according to the present invention does not tend to swell.

Thus, based on this example it is demonstrated that the cereal straw product according to the present invention is suitable as a diet product and may be suitable for the treatment or reduction of obesity.

### Example 2

Recipes where Rye Flour May be Substituted with Wheat Straw According to the Present Invention.

6 different breads were made. The breads had the following ingredients, listed as wight of the ingredient by weight of the unbaked dough:

| | 1 | 2 | 3 | 4 | 5 | 6 |
|---|---|---|---|---|---|---|
| Water | 43% | 43% | 43% | 43% | 40% | 40% |
| Rye flour | 24% | 23% | 19% | 16% | 0 | 0 |
| Malt flour | 3% | 3% | 3% | 3% | 0 | 0 |
| Wheat | 0 | 0 | 0 | 0 | 40% | 37% |
| flour | | | | | | |
| Duorum | 0 | 0 | 0 | 0 | 30% | 30% |
| flour | | | | | | |
| Sourdough | 4% | 4% | 4% | 4% | 4% | 4% |
| Wheat | 0 | 1% | 5% | 8% | 0 | 3% |
| straw | | | | | | |
| Oatmeal | 5% | 5% | 5% | 5% | 5% | 5% |
| Cereal | 21% | 21% | 21% | 21% | 11% | 11% |
| seeds* | | | | | | |
| Total | 100% | 100% | 100% | 100% | 100% | 100% |

*The cereal seeds comprise a mixture of cereal seeds, like pumpkin seeds; sunflower seeds; and broken rye kernels.

Bread 1-4 are recipes for rye bread where ray flour is stepwise substituted by 1-8% (w/w) wheat straw. Bread 5-6 are recipes for white bread where ray flour is substituted by 3% (w/w) wheat straw.

The ray breads were baked at 200° C. for 65 minutes and allowed to cool and settle for at least 2 hours. The white breads were baked at 220° C. for 35 minutes and allowed to cool for 2 hours.

The breads (both the ray bread and the white bread) showed a homogenous and coherent construction when cut.

The breads with and without wheat straw, showed to have a crispy crust and juicy crumb, and the aroma and taste were slightly increasing with increasing content of wheat straw, and the umami taste was increased with increasing content of wheat straw.

The shelf-life of the bread was also increasing with increasing content of wheat straw in the bread dough.

### Example 3

### Investigating the Effects of a Cereal Straw Product According to the Present Invention Intestinal Malfunctioning and Stomach Pain.

Test-tablets comprising 1 g wheat straw product having a particle size d50 of about 1 mm were produced according to the process described in Example 1.

A female patient, 55 years old, was suffering from intestinal malfunctioning and stomach pain, She was administered 5 Psyllium Husk capsules (each of 500 mg per capsule) 2 times a day (one time in the morning and one time in the evening) for 8 weeks. After 8 weeks there were no sign of improvements, and the patient was signed up for a stoma surgery.

The patient started taken test-tablets according to the present invention, the test tablets was taken 3 times a day and the dose was 5 gram per dose. Thus, the daily dose was 15 g per day.

After 6 weeks the intestine was working normally, the stomach pain was gone, and the stoma surgery was cancelled.

Furthermore, the patient also found that the nauseas and swollen feeling experienced during the period of administering Psyllium Husk capsules disappeared when changing to the administration of the test-tablets according to the present invention.

### Example 4

### Investigating the Effects of a Cereal Straw Product According to the Present Invention on Weight Loss and Better Gut Feeling.

The patient was administered 6 g per day in six months in a smoothie in the morning.

The male patient lost 1 kg/months in weight, in total 6 kg, and the BMI fell from 36 to 34.

The patient experienced higher satiety, had no sudden hunger, and had lower intake of food. The patient had less bloating and flatulence and went from irregular defecation to regular stool of normal and consistent consistence.

The general wellbeing improved due to less bloating.

### Example 5

### Investigating the Effects of a Cereal Straw Product According to the Present Invention on Inflammatory Bowel Disease (IBD).

The patient was administered 8 g per day for two months in a smoothie in the morning.

The female patient had a BMI of 21 at the beginning and did not register any weight loss.

The patient experienced significant higher satiety and a longer duration of the sensation, and had a clearly lower intake of food. The satiety was whole morning and she felt less hunger at lunch. The patient, instead of eating a snack after 2 hours and eating 300 gram whole grain rye bread at lunch, during the test had no need for snacks, and the lunch was half dose of the usual.

Before the treatment, the patient had hard stool and defecation once a week; during intake period, defecation was daily and stool normal.

Regarding general well-being, there were no symptoms of IBD during the trial, the IBD condition of bloating and stomach pain disappeared, and she had a feeling of more energy.

### Example 5

### Investigating the Effects of a Cereal Straw Product According to the Present Invention on IBD and Overweight

The patient was administered 10 g per day in twelve months in a smoothie in the morning.

The female patient lost 1 kg/months in weight, in total 13 kg. The patient's BMI fell from 28 to 24.

The patient experienced higher satiety, the usual time to craving after breakfast of 2 hours and need to have a snack, was significantly increased to 4-5 hours during intake. The satire sensation is not that of being complete full, it is a present calm satiety.

The patient had less bloating, no bowel pains and no flatulence. Usual constipation was completely relieved. The pre intake occurrence of defecation was once a week. During intake, defecation was daily and normal consistency. No diarrhea and no constipation.

Regarding wellbeing, the stomach feels soft, and the general feeling of the body is relaxed and calm.

1-24. (canceled)

25. A cereal straw product for human or non-ruminant administration, comprising a cereal straw, wherein at least 50% of the cereal straw has a size (d50) less than 5 mm and/or wherein at least 90% of the cereal straw has a size (d90) less than 8 mm, wherein the cereal straw product comprises less than 15% (w/w) short chain carbohydrate.

26. The cereal straw product according to claim 25, comprising a cereal straw, wherein the size (d50) is in the range of 5-0.005 mm, such as in the range of 4-0.01 mm, e.g. in the range of 3-0.05 mm, such as in the range of 2-0.1 mm, e.g. in the range of 1-0.5 mm, and/or the size (d90) is in the range of 8-0.005 mm, such as in the range of 5-0.01 mm, e.g. in the range of 3-0.05 mm, such as in the range of 2-0.1 mm, e.g. in the range of 1.5-0.5 mm.

27. The cereal straw product according to claim 25, comprising at least 50% (w/w) cereal straw relative to the dry weight of the cereal straw product, such as at least 60% (w/w), e.g. at least 70% (w/w), such as at least 75% (w/w), e.g. at least 80% (w/w), such as at least 85% (w/w), e.g. at least 90% (w/w), such as at least 95% (w/w), e.g. at least 98% (w/w), such as 100% (w/w).

28. The cereal straw product according to claim 25, wherein the cereal straw product comprises less than 15% (w/w) short chain carbohydrate.

29. The cereal straw products according to claim 25, selected from wheat straw, oat straw, barley straw, rye straw, triticale straw, or a combination hereof.

30. The cereal straw product according to claim 25, wherein the cereal straw product is substantially free from cereal grains or cereal bran.

31. The cereal straw product according to claim 25, wherein the cereal straw product comprises at least 95% (w/w) cellulose compounds; e.g., at least 98% (w/w) cellulose compounds, such as at least 99% (w/w) cellulose compounds.

32. The cereal straw product according to claim 25, wherein the cereal straw product consists essentially of cellulose compounds, hemicellulose compounds, and lignin.

33. The cereal straw product according to claim 25, wherein the cereal straw has not been subjected to a pre-treatment, such as a hydrolysing treatment, a chemical treatment, an enzymatic treatment, a heat treatment, a pressure treatment, a fermentation treatment, or a combination hereof.

34. A composition comprising a cereal straw product according to claim 25.

35. The composition according to claim 34, wherein the composition comprises in the range of 0.5-100% (w/w) of the cereal straw product relative to the dry weight of the composition, such as in the range of (0.75-95% (w/w), e.g. in the range of 1-85% (w/w), such as in the range of (1.25-75% (w/w), e.g. in the range of 1.5-50% (w/w), such as in the range of 1.75-25% (w/w), e.g. in the range of 2-15% (w/w), such as in the range of 2.5-10% (w/w), e.g. in the range of 3-8% (w/w), such as in the range of 3.5-6% (w/w), e.g. in the range of 4-5% (w/w).

36. The composition according to claim 34, wherein the composition is a diet product, a flour product, an ingredient, and/or an absorbing product.

37. The composition according to claim 34, wherein the administration form is oral or rectal.

38. The composition according to claim 34, wherein the composition is formulated as a powder, as a drink, as a capsule, or as a tablet.

39. A tablet or a capsule comprising a cereal straw product according to claim 25 and optionally a binder ingredient.

40. Cereal straw product according to claim 25 for use in the treatment or prophylaxis of the human or animal body.

41. The cereal straw product according to claim 40 for use in the treatment or prophylaxis of conditions related to the gastrointestinal system, such as obesity, overweight, diarrhea, bloating, flatulence (stomach or bowl gas), inflammatory bowel disease (IBD), Crohn's disease, pain (stomach pain), cholesterol levels, heart disease, type 2 diabetes, or colon cancer and/or for the treatment of intestinal induced diseases and/or normalisation of the intestinal microbiota.
